(19) United States
(12) Patent Application Publication
(10) Pub. No.: US 2009/0118626 A1
(43) Pub. Date: May 7, 2009

(54) Calculating Respiration Parameters Using Impedance Plethysmography

(75) Inventors: Loell Boyce Moon, Ham Lake, MN (US); Paul Haefner, Circle Pines, MN (US); Andres Belalcazar, Saint Paul, MN (US)

Correspondence Address: FISH & RICHARDSON P.C., P.O. BOX 1022, MINNEAPOLIS, MN, 55440-1022, US

(73) Assignee: Transoma Medical, Inc., Arden Hills, MN (US)

(21) Appl. No.: 11/933,872

(22) Filed: Nov. 1, 2007

(51) Int. Cl. A61B 5/0205

(52) U.S. Cl. 600/484

(57) ABSTRACT

A method of determining a value for a respiration parameter in a test subject can include capturing—using a fully implanted system that includes a wireless transmitter and at least a first lead wire having a first electrode disposed thereon and a second lead wire having a second electrode disposed thereon—information indicative of an impedance measure between the first and second electrodes and across a thoracic region of the test subject; wirelessly transmitting, from the implanted system and to external equipment, the captured information; and determining a respiration parameter of the test subject based on the captured information. The at least first and second lead wires can be positioned in the test subject subcutaneously and external of any cranial, thoracic, abdominal and pelvic cavities of the test subject

### BACKGROUND

Animal testing is a critical component of preclinical testing of new pharmaceutical compounds that ultimately may be approved for therapeutic use by human patients. In particular, animal testing can be used to initially assess pharmacodynamics, pharmacokinetics and toxicity of a compound. Based on the animal testing, some compounds may be tested in human clinical trials.

To initially assess pharmacodynamics, pharmacokinetics and toxicity of a compound, the compound may be administered in a controlled manner to laboratory animals (e.g., test subjects, such as mice, rats, guinea pigs, dogs, etc.), and the laboratory animals can be subsequently monitored. Of the various physiological parameters that are frequently monitored during testing, several parameters may be particularly important. For example, the International Conference on Harmonization of Technical Requirements for Registration of Pharmaceuticals for Human Use (ICH)—a group that brings together regulatory authorities in the United States, Europe and Japan for the purpose of harmonizing regulatory guidelines for testing and approving new pharmaceutical compounds—has identified cardiovascular, respiratory and central nervous systems as particularly important. Specifically, the ICH, in its S7A Safety Pharmacology Studies for Human Pharmaceuticals guidelines, has included cardiovascular, respiratory and central nervous systems in a core battery that should be evaluated prior to the first administration of a pharmaceutical substance in humans.

To evaluate the likely effect of a pharmaceutical compound on cardiovascular, respiratory and central nervous systems of humans, the pharmaceutical compound may be tested in various animal models, and various physiological parameters of the animals models may be monitored during the testing. For example, an electrocardiogram (ECG) signal, blood pressure and blood flow rate can be monitored to evaluate the effect of a compound on the cardiovascular system. As another example, motor activity can be monitored (e.g., with electromyography (EMG) parameters), changes in behavior or coordination can be noted, sensory and motor reflex responses can be tracked (e.g., with electroencephalography (EEG) parameters, EMG parameters, or electrooculography (EOG) parameters), and internal body temperature can be monitored to evaluate the effect of a compound on the central nervous system. As another example, respiratory flow, tidal volume, hemoglobin oxygen saturation, and other respiratory parameters can be monitored to evaluate the effect of a compound on the respiratory system.

Various devices can be employed to monitor respiration parameters. For example, a plethysmography chamber can be used to measure respiratory flow of a restrained test subject, such as a laboratory rat, over a period of an hour or two. In some such chambers, the test subject is restrained at the neck and fitted with a hood that is configured with a precise airflow monitoring system. In other chambers, animals are permitted a small amount of movement within a small enclosure that is also configured with a precise airflow monitoring system. Respiration parameters can also be obtained from anesthetized animals with a breathing tube fitted with precise pressure or flow sensors. In addition, jacket-based systems can allow certain respiration parameters to be gathered from cooperative animals over a period of one or two days.

### SUMMARY

During preclinical testing of pharmaceutical compounds on test subjects (or in other research studies of the effect of other test substances on test subjects), various physiological parameters of the test subjects can be monitored with a wireless implantable device. The wireless implantable device can facilitate collection of physiological data from unrestrained and unanesthetized test subjects. In particular, respiration parameters can be obtained in a minimally invasive manner, using subcutaneously implanted electrodes. More specifically, time-varying thoracic impedance values can be obtained, from which tidal volume, respiratory rate, inspiratory time or interval and flow, and expiratory time or interval and flow can be determined. When other sensors are also implanted, data from the other sensors can be combined with some of the above-mentioned respiration parameters to obtain additional respiration parameters, such as, for example, a test subject's lung compliance or a test subject's airway resistance.

In some implementations, a method of determining a value for a respiration parameter in a test subject can include capturing—using a fully implanted system that is comprised of a wireless transmitter and at least a first lead wire having a first electrode disposed thereon and a second lead wire having a second electrode disposed thereon, wherein the at least first and second lead wires are positioned in the test subject subcutaneously and external of any cranial, thoracic, abdominal and pelvic cavities of the test subject—information indicative of an impedance measure between the first and second electrodes and across a thoracic region of the test subject; wirelessly transmitting, from the implanted system and to external equipment, the captured information; and determining a respiration parameter of the test subject based on the captured information.

The respiration parameter may be tidal volume. Determining the tidal volume may include determining tidal volume in the external equipment, based on the wirelessly transmitted information. Capturing the information indicative of the impedance measure may include capturing the information when the test subject is unrestrained and unanesthetized. The determined respiration parameter may be used in safety pharmacology testing or toxicity testing to assess impact of a pharmaceutical compound on the test subject's respiratory system.

In some implementations, the electrodes are positioned in the test subject such that the impedance measure between the electrodes is made across the thoracic region of the test subject in manner that crosses a sagittal plane of the test subject in a cranial-to-caudal or posterior-to-inferior manner. In some implementations, the electrodes are positioned in the test subject such that the impedance measure between the electrodes is made through at least a portion of one of the test subject's lungs.

The transmitter may be positioned subcutaneously and external of any cranial, thoracic, abdominal and pelvic cavities of the test subject. Capturing information indicative of the impedance measure may include injecting a current between two electrodes and measuring a resulting voltage between two electrodes.

In some implementations, the fully implanted system includes four electrodes, and the two electrodes between which current is injected and the two electrodes between which the resulting voltage is measured are each distinct electrodes. The captured information may include a value for the measured resulting voltage.

In some implementations, the test subject is selected from the group of laboratory animals consisting of rodents, bovines, canines, ovines, porcines and non-human primates. In other implementations, the test subject is a human patient.

The impedance measure may include a base impedance component and a modulated impedance component. Determining the respiration parameter may include normalizing the modulated impedance component relative to the base impedance component. Determining the respiration parameter of the test subject based on the captured information may include identifying in the captured information first periodic data having a first dominant frequency and second periodic data having a second dominant frequency that is lower than the first frequency, and determining the respiration parameter of the test subject based on the second periodic data.

The fully implanted system may include a pressure sensor configured to obtain blood pressure information from the test subject; identifying in the captured information the first periodic data may include identifying the first periodic data based on the blood pressure information. The fully implanted system may include a sensor configured to obtain electrocardiogram (ECG) data from the test subject; identifying in the captured information the first periodic data may include identifying the first periodic data based on the ECG data. The fully implanted system may include a pressure sensor, and the method may further include capturing, with the pressure sensor, information indicative of an internal pressure of the test subject.

In some implementations, the method further includes identifying in the captured information third data having spectral content that indicates that the third data has been corrupted by at least one of the test subject's posture or the test subject's activity, and removing the third data from the captured information. The fully implanted system may include an accelerometer sensor, and identifying in the captured information the third data may include identifying the third data based on data from the accelerometer sensor. The fully implanted system may include an electromyogram (EMG) sensor, and identifying in the captured information the third data may include identifying the third data based on data from the EMG sensor.

The method may further include measuring tidal volume of the test subject with a device that is external to the test subject, and calibrating a relationship between the captured information and the measured tidal volume. Determining the value for the tidal volume of the test subject may include correlating the captured information with the tidal volume based on the calibrated relationship. The calibrated relationship may be a function of mass of the test subject, and the method may further include normalizing the tidal volume based on a mass of the test subject.

Determining the value for the respiration parameter of the test subject may include identifying a time-ordered sequence of impedance values in the captured information, and determining, based on the time-ordered sequence of impedance values, at least one of a tidal volume, an inspiratory time, an expiratory time, an inspiratory flow or an expiratory flow of the test subject.

In some implementations, an implantable device configured to monitor thoracic impedance in a test subject includes a fully implantable system having a controller and at least a first lead wire and a second lead wire. The controller and the first and second lead wires may be configured to be subcutaneously implanted in the test subject, external of any cranial, thoracic, abdominal or pelvic cavities of the test subject. The first lead wire may have first and second electrodes disposed thereon and the second lead wire may have third and fourth electrodes disposed thereon. The controller may include a) a current signal generator that generates a current signal between the first and third electrodes; b) a voltage amplifier that detects a voltage difference between the second and fourth electrodes; and c) a wireless transmitter that is configured to transmit information to external equipment when the test subject is ambulatory and unanesthetized. The information may include the detected voltage difference or a signal derived from the detected voltage difference.

The details of one or more implementations are set forth in the accompanying drawings and the description below. Other features, objects, and advantages will be apparent from the description and drawings, and from the claims.

### DESCRIPTION OF DRAWINGS

FIG. 1 is a diagram of an example environment in which an implantable monitoring device may be used.

FIG. 2A is a block diagram of an example implantable monitoring device.

FIGS. 2B and 2C are block and schematic diagrams, respectively, of an impedance sensor that can be included in an implantable monitoring device.

FIG. 2D illustrates various example configurations of lead wires that can be used to monitor impedance.

FIG. 3 is an illustration depicting how the device shown in FIG. 2 may be implanted in a laboratory animal.

FIGS. 4A-4D are diagrams illustrating various anatomical aspects and details that are discussed with reference to implantable monitoring devices.

FIGS. 5A and 5B illustrate example electrode positions for monitoring thoracic impedance in two test subjects.

FIG. 6A graphically depicts data that can be received from an implantable monitoring device.

FIG. 6B graphically depicts a cardiac signal that may be included in the data depicted in FIG. 6A.

FIG. 6C graphically depicts the data from FIG. 6A after the cardiac signal shown in FIG. 6B has been removed.

FIG. 7 is a flow diagram of an example method of obtaining respiration-based thoracic impedance data.

FIG. 8 illustrates example thoracic impedance data obtained from an implantable device and corresponding measured respiration data from an external device.

FIG. 9 graphically illustrates an example method of calibrating an impedance-tidal volume relationship.

FIG. 10A is a flow diagram of an example method of determining a tidal volume of a test subject based on a calibrated relationship between impedance and tidal volume.

FIG. 10B a flow diagram of an example method of calibrating a relationship between tidal volume and change in thoracic impedance.

FIG. 11 illustrates a time-varying sequence of derivatives of a corresponding time-varying sequence of tidal volume values, which can be used to determine additional respiration parameters.

FIG. 12 illustrates three example lung compliance curves.

Like reference symbols in the various drawings indicate like elements.

### DETAILED DESCRIPTION

During preclinical testing of pharmaceutical compounds on test subjects (or in other research studies of the effect of other test substances on test subjects), various physiological parameters of the test subjects can be monitored with a wireless implantable device. The wireless implantable device can facilitate collection of physiological data from unrestrained and unanesthetized test subjects. In particular, respiration parameters can be obtained in a minimally invasive manner, using subcutaneously implanted electrodes. More specifically, time-varying thoracic impedance values can be obtained, from which tidal volume, respiratory rate, inspiratory time or interval and flow, and expiratory time or interval and flow can be determined. When other sensors are also implanted, data from the other sensors can be combined with some of the above-mentioned respiration parameters to obtain additional respiration parameters, such as, for example, a test subject's lung compliance or a test subject's airway resistance.

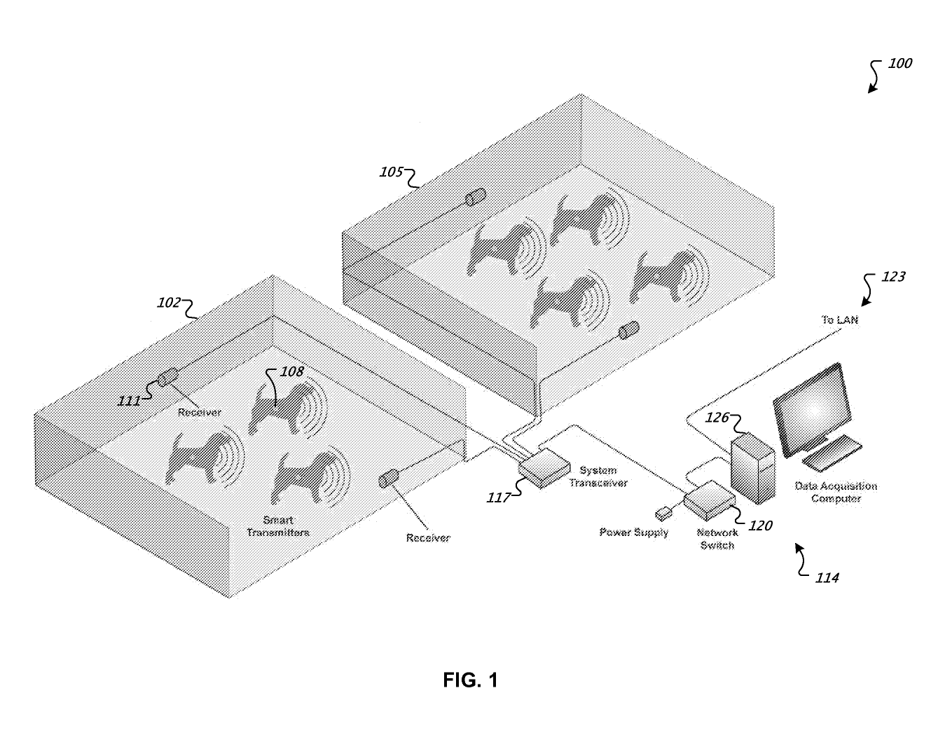

FIG. 1 illustrates one example environment 100 in which physiological parameters of test subjects (e.g., laboratory animals) can be captured with an implanted device in a controlled environment (e.g., in the context of preclinical testing of pharmaceutical components). In FIG. 1, the test subjects depicted are dogs; however, the environment 100 can be used to monitor physiological parameters of any kind of laboratory animal. As shown in one implementation, the environment 100 includes containment areas 102 and 105. Each containment area 102 or 105 can be configured to house multiple animals, as shown (e.g., to permit natural social interaction between the animals); alternatively, containment areas can be configured to house a single animal.

As depicted in one implementation, a monitoring device, such as the monitoring device 108, is implanted in each animal. The monitoring device 108 (or implantable device 108) can include one or more sensors configured to capture one or more physiological parameters of the animal, and a transmitter configured to transmit captured physiological parameters to a receiver, such as the receiver 111 (which, in some implementations, may be replaced by a transceiver). As shown, various receivers are located in the containment areas. Each receiver is connected to an acquisition system 114, which can receive, store and analyze physiological data. In one implementation, as shown, various receivers are connected to a system transceiver 117, which can combine data received from multiple receivers into a single data stream (or smaller number of data streams). The acquisition system 114 can include network connections, such as a network switch 120 or LAN connection 123, to permit the system to monitor a larger number of containment areas or to facilitate remote access of data. The acquisition system 114 can include a storage and analysis device, such as a computer 126, which can be used to receive, store, display and analyze captured physiological data.

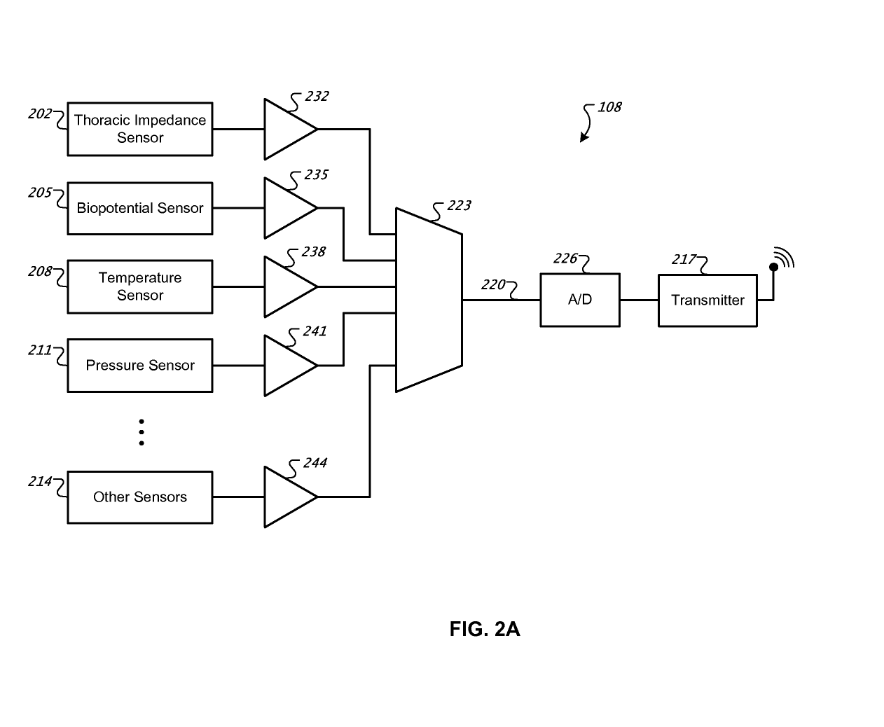

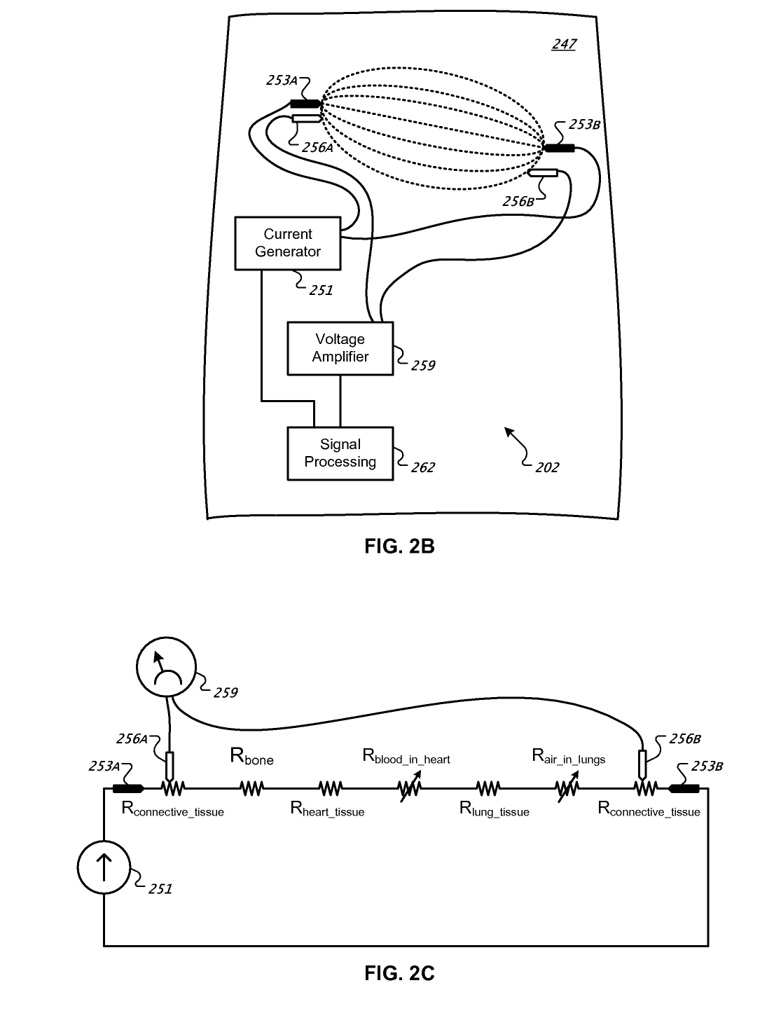

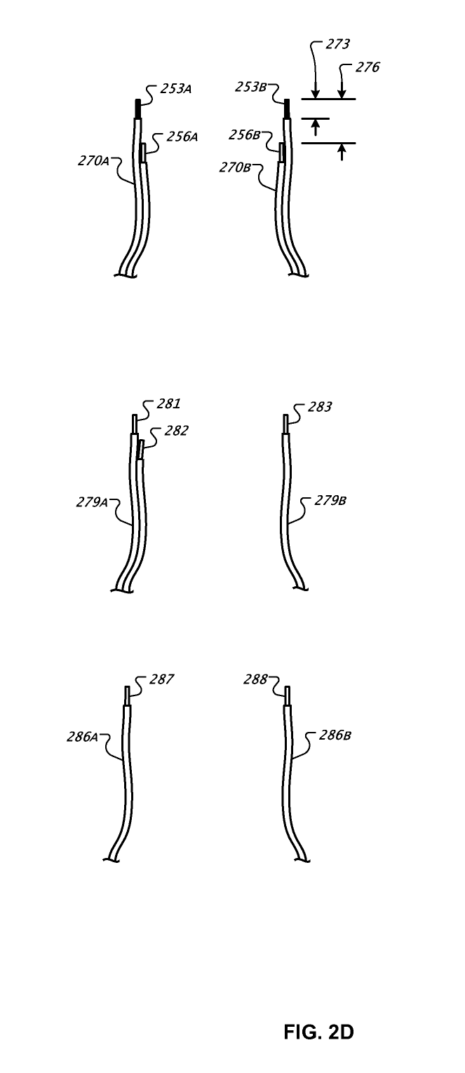

FIG. 2A illustrates additional details of the example implantable device 108 that is depicted in FIG. 1. As described above, the implantable device 108 can include a number of sensor devices that can be implanted in a test subject. The sensor devices can include, for example, a thoracic impedance sensor 202, which is described in greater detail with reference to FIG. 2B; a biopotential sensor 205 (e.g., a sensor for measuring biopotential signals, such as electroencephalography (EEG) signals, electrocardiogram (ECG) signals, electromyography (EMG) signals, or electrooculography (EOG) signals); a temperature sensor 208; a pressure sensor 211 (e.g., for sensing blood pressure or pressure of an internal cavity); and other sensors 214.

Other sensors 214 can include, for example, an accelerometer, which can be used to detect position, movement or behavior of a test subject. Any other sensor configured to monitor a physiological parameter of a test subject can be included in the implantable device 108. In particular, some implantable devices 108 include a suite of sensors that enable researchers to obtain a large amount of data (e.g., data that is typically collected during pharmaceutical testing) with a single device. For example, a suite of sensors could include one or more of the following: blood flow sensors, edema sensors, ionic state sensors (e.g., for sensing K⁺, NA⁺, CA⁺), gas sensors (e.g., for sensing NO, O, O₂, or CO₂), pH sensors, glucose sensors, insulin sensors, oxygen saturation sensors, various pressure sensors, posture and activity sensors, sound sensors (e.g., for heart sound or rales detection), etc.

Values of physiological parameters captured by the various sensors 202-214 can be transmitted by a transmitter 217 to an external system, such as the receiver 111 and acquisition system 114 (external system 114) shown in FIG. 1. As shown in one implementation, values of the physiological parameters can be multiplexed into a single signal 220 with a signal combiner 223 (e.g., a multiplexer), and the signal 220 can be converted from an analog format to a digital format with an analog-to-digital converter 226 (A/D 226) before being transmitted.

In some implementations, signals from various sensors can be amplified, or the signals can otherwise be processed (e.g., with amplifiers 232-244). Gain may be individually configurable for each sensor 202-214, and in some implementations, filtering may be applied to individual or multiple signals. For example, the amplifier 235 may include a filter (not explicitly shown) to filter ECG signals captured by the biopotential sensor 205 out of the signal that is captured by the thoracic impedance sensor 202.

The A/D function is shown for purposes of example as following the multiplexer 223, but in some implementations, signals are digitized before being multiplexed. In other implementations, signals may be transmitted in analog form (e.g., encoded in a form that permits an analog representation (e.g., analog frequency modulation, amplitude modulation, pulse width modulation, pulse position modulation, etc.)).

In some implementations, each sensor signal is allotted a timeslot, such that the resulting signal 220 is a time-division multiplexed signal. For example, the signal 220 could be formatted into frames with a number of timeslots, and each sensor could provide data for a particular timeslot in each frames.

FIGS. 2B and 2C are a block diagram and a schematic diagram, respectively, illustrating additional details of the example thoracic impedance sensor 202. The thoracic impedance sensor 202 can be used to measure thoracic impedance in body tissue 247 of a test subject. In operation, the thoracic impedance sensor 202 can detect changes in thoracic impedance resulting from physiological changes. Bone, organ tissue (e.g., tissue of the heart and lungs) and connective tissue present a relatively constant impedance (as depicted by the fixed resistance in the schematic diagram shown in FIG. 2C); air is highly resistive, and ionized fluids (e.g., blood) have a low resistance. Accordingly, variations in air volume and changes in blood flow can directly cause changes in transthoracic impedance (as indicated by the variable resistances in FIG. 2C). A direct correlation has been established between changes in thoracic impedance during respiration cycles and the tidal volume of air inhaled and exhaled during the respiration cycles. Accordingly, tidal volume, and various other parameters that can be derived from a tidal volume-time function, can be obtained from measurements of thoracic impedance.

In one implementation as shown, the thoracic impedance sensor 202 includes a current generator 251 that generates a current signal, which passes through body tissue 247 of the test subject from an electrode 253A to an electrode 253B. The current signal passes between the electrodes along many different paths and through different body tissues and structures. The amplitude of the signal is modulated by changes in thoracic impedance, which in many implementations, results from changes in air volume in the lungs and blood volume in the heart. The modulation of the current signal can be detected as a change in potential difference between different points in the body tissue 247. Put another way, a time-varying voltage can be detected in the body tissue 247, and the magnitude of the time-varying voltage is related to the magnitude of the original current signal, the base thoracic impedance, and the change in thoracic impedance caused by respiration and other physiological processes (e.g., blood flow variations related to cardiac function). A separate set of electrodes 256A and 256B and a voltage amplifier 259 (e.g., one or more field effect transistors (FETs) and a differential amplifier, in one implementation) can detect the voltage difference created by the current signal and the impedance of the body tissue 247 along a path between the voltage electrodes 256A and 256B.

In some implementations, a separate signal processing element 262 converts the detected voltage to an impedance (e.g., by dividing the magnitude of the detected voltage by the magnitude of the current signal). In other implementations, the voltage signal is maintained as such, and the conversion to an impedance value can be performed elsewhere in the system (e.g., in the external system 114). Other signal processing may be performed by the signal processing element 262. For example, the signal processing element 262 may filter the signal (e.g., to remove noise at a particular frequency or range of frequencies; more particularly, some implementations employ a band-pass filter having a center frequency at the frequency of the current signal), or the signal processing element 262 may digitize the detected voltage or calculated impedance value.

The current signal can be any signal that will create a detectable voltage signal without causing other adverse effects (e.g., muscle sensation or stimulation, pain, tissue destruction, etc.). Frequently, the current signal is a very low, periodic current signal. For example, the current signal can be a sinusoidal or pulsed signal having a frequency of 1-100 kHz (e.g., 25 kHz) and an amplitude of 50-400 uA (e.g., 200-300 uA). In some implementations, a pulsed (e.g., square wave) signal may be preferred over other signals because a pulsed signal can be easy to generate and may also require less power than, for example, a sinusoidal signal. Other implementations employ other kinds of signals (e.g., triangle, bi-phasic, etc.), and may employ other frequencies or amplitudes.

In some implementations, various parameters may be adjustable or programmable, either manually (e.g., remotely, from signals transmitted from the external system 114 to a receiver and corresponding processing circuitry (not shown) in the implantable device 108). In particular, for example, amplitude or frequency of the current signal generated by the current generator 251 may be adjustable (e.g., to facilitate use of the implantable device 108 in test subjects of various sizes). As another example, the gain for individual sensors (e.g., the gain of amplifiers 232-244) may be adjustable (e.g., manually, or automatically-based on processing circuitry internal to the implantable device 108) to facilitate a high signal-to-noise ratio in a variety of operating environments. In some implementations, frequency and current amplitude are both adjustable to maximize the signal-to-noise ratio while minimizing power consumption.

In FIG. 2B, four discrete electrodes 253A, 253B, 256A and 256B are shown (a tetrapolar lead arrangement). Additional details of such a tetrapolar arrangement are shown in with reference to FIG. 2D. In particular, in one implementation as shown, the four electrodes 253A, 253B, 256A and 256B are disposed on two lead wires 270A and 270B-two electrodes on each lead wire. In some implementations, for example implementations in which the lead wires 270A and 270B and corresponding electrodes are implanted in small animals (e.g., rodents), the electrodes may have an approximate length 273 of ½ cm (e.g., lead exposure, or electrode length), and a distance 276 of approximately 1 cm may separate multiple electrodes (e.g., electrodes 253B and 256B) on a single lead wire (e.g., lead wire 270B). In other implementations, electrode lengths and distances between electrodes on a single wire can have different dimensions. For example, in larger animals (e.g., canines), electrodes may have an approximate length 273 of 1 cm, and a distance 276 of approximately 5 cm.

In general, the dimensions of the electrodes (e.g., electrode length and electrode separation) can be optimized to capture a good signal, based, for example, on the size of the test subject. In particular, for example, the closer the electrodes 253B and 256B are (e.g., in a tetrapolar configuration), the more artifacts (e.g., from movement) that may be picked up. As electrodes 253B and 256B are separated, the signal may improve. In addition, distance between the electrodes 253B and 256 can control the depth of the impedance measurement (that is, a greater separation can facilitate a more deep impedance measurement in the test subject than a smaller separation). Amplitude of the current signal can also affect signal quality (e.g., signal-to-noise ratio). Thus, amplitude of the current can be increased for larger animals, within constraints imposed, for example, by power consumption requirements and limits on current to prevent tissue from being stimulated.

In many tetrapolar implementations, electrodes on the same lead wire are configured to remain a fixed distance from each other following implantation. In particular, for example, the electrodes 253B and 256B may be rigidly fixed relative to each other to prevent changes in the detected voltage signals once the lead wires 270A and 270B are implanted in the test subject.

The electrodes themselves can be made of any material that is suitable for implantation in a living being. For example, some electrodes are made of bare wire formed from or coated with a gold or titanium alloy. In some implementations, the electrodes are merely exposed portions of the lead wires. In other implementations, the electrodes are separately formed (e.g., to increase their surface area or to provide a custom shape) and attached to corresponding lead wires. Electrodes may be coated to enhance signal pickup, minimize corrosion or chemical or ion interaction with the tissue, or prevent tissue from sticking to or growing onto the electrodes. In particular, for example, some electrodes are coated with polytetrafluoroethylene (PTFE). As another example, some electrodes are coated with platinum black.

The lead wires 270A and 270B are depicted as parallel, two-conductor lead wires, but in other implementations, the lead wires can have different arrangements. In particular, for example, multi-conductor lead wires can have a co-axial or co-radial arrangement. The conductors within various kinds of lead wires can be cylindrical or flat.

Once implanted, lead wires can be anchored in various manners in a test subject—for example, to control the depth and orientation of the current field. In particular, the lead wires can be directly sutured (e.g., with the aid of tabs) to skin or muscle of the test subject, or the lead wires can be threaded though a sleeve which is itself sutured to the skin or muscle of the test subject. Alternatively, a mesh (e.g., a Dacron™ mesh—not shown in FIG. 2D) can be provided to serve as an anchor surface on the end of a lead wire. Other known anchoring techniques can be employed to prevent a lead wire from moving in an undesirable manner once it is implanted.

Other configurations of lead wires and electrodes are shown in FIG. 2D. In particular, for example, lead wires 279A and 279B illustrate a tripolar arrangement in which two electrodes 281 and 282 are disposed on one lead wire 279A and a third electrode 283 is disposed on the second lead wire 279B. In a tripolar arrangement a current signal can be provided between electrodes 281 and 283, and a voltage signal can be sensed between electrodes 282 and 283. In such an arrangement, the electrode 283 can be common to both the current-generating and voltage-sensing circuits. In a bipolar implementation, both electrodes 287 and 288 can be common to the current-generating and voltage-sensing circuits, and each electrode can be disposed on its own respective lead wire 286A or 286B. Other configurations are possible. For example, by employing greater numbers of electrodes, thoracic impedance measurements could be captured from a number of different regions in the test subject.

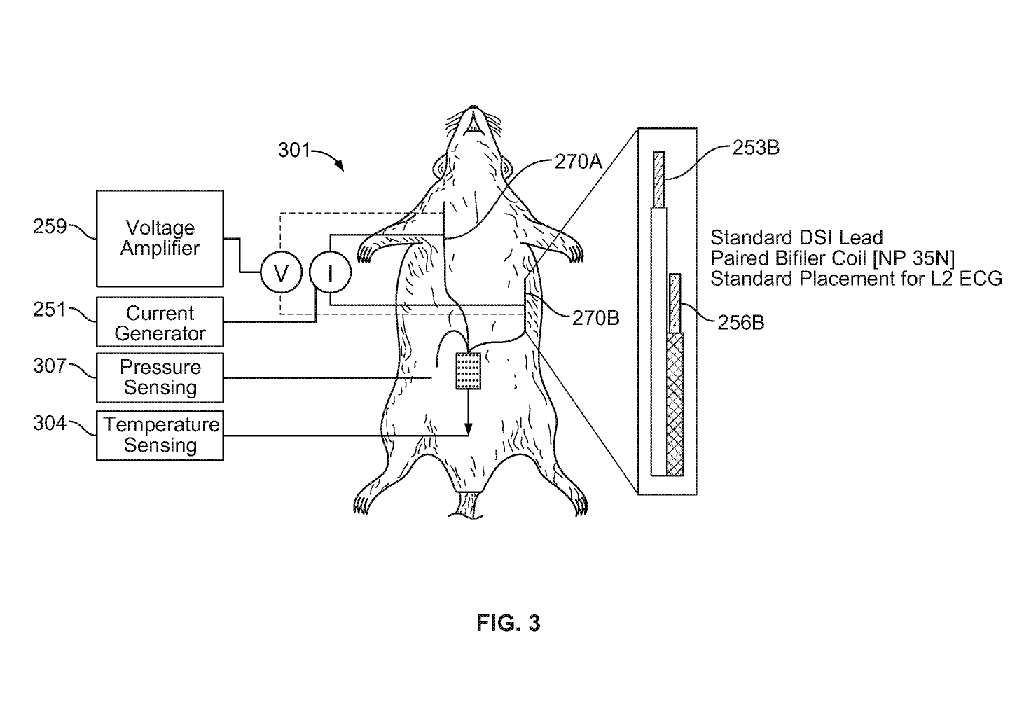

Although described above in the context of measuring thoracic impedance, the lead wires (e.g., lead wires 270A and 270B) for measuring thoracic impedance can also be used to capture other biopotential information. In particular, for example, the electrode 256B (shown in FIG. 2D and FIG. 3) can be used to capture one ECG signal, and the electrode 256A can be used to capture another ECG signal (e.g., another standard channel of single-ended ECG information). Alternatively, the electrodes 256A and 256B can together provide a differential biopotential signal. In some implementations, the biopotential signals are captured at substantially the same time that thoracic impedance values are obtained (e.g., signals on the appropriate electrodes may be sampled at some frequency, and the samples may alternate between sampling thoracic impedance information (e.g., voltage induced by the above-described current signal) and sampling ECG information (e.g., each sample or based on some other pattern, such as one thoracic impedance sample for every five ECG samples). In such implementations, the ECG (or other biopotential information) may be sampled in a manner that is synchronized with the current signal (e.g., such that the sample is made when the current generator is not actively providing current to the body tissue, such as the off portion of a pulsed current signal). In other implementations, the leads may be used for either capturing ECG or other biopotential information, or for capturing thoracic impedance information, and the current function of the leads may be remotely programmable or adjustable. Other configurations and measurements are contemplated. For example, all four electrodes 253A, 253B, 256A and 256B in the tetrapolar configuration shown in FIG. 2D could be employed to capture biopotential information.

FIG. 3 is a diagram of the implantable monitoring device 108, shown implanted in a laboratory rat 301. For purposes of example, a tetrapolar lead arrangement is depicted, and a temperature sensor 304 and pressure sensor 307 are also shown to be included in the device and implanted in the laboratory rat 301. In one implementation as shown, pairs of electrodes are physically disposed in the two lead wires 270A and 270B. In this example, as described with reference to FIGS. 2B and 2C, a current signal is propagated from a first electrode 253A (not labeled in FIG. 3) on a first lead wire 270A to a second electrode 253B on a second lead wire 270B, and a resulting voltage difference is detected between a third electrode 256A (not labeled in FIG. 3) on the first lead wire 270A and a fourth electrode 256B on the second lead wire 270B. The current generator 251 and voltage amplifier 259 are depicted outside of the laboratory rat 301 for clarity, but the reader will appreciate, in light of the above description with reference to FIGS. 1 and 2A-2D, that the current generator 251, voltage amplifier 259 and other components can be fully implanted in the laboratory rat 301.

Placement of the lead wires can impact the quality of the sensed thoracic impedance. A detailed discussion of sensor and lead wire placement is provided with reference to FIGS. 5A and 5B, which follows a brief discussion of anatomy to clarify and inform the discussion of placement using standard anatomical terms.

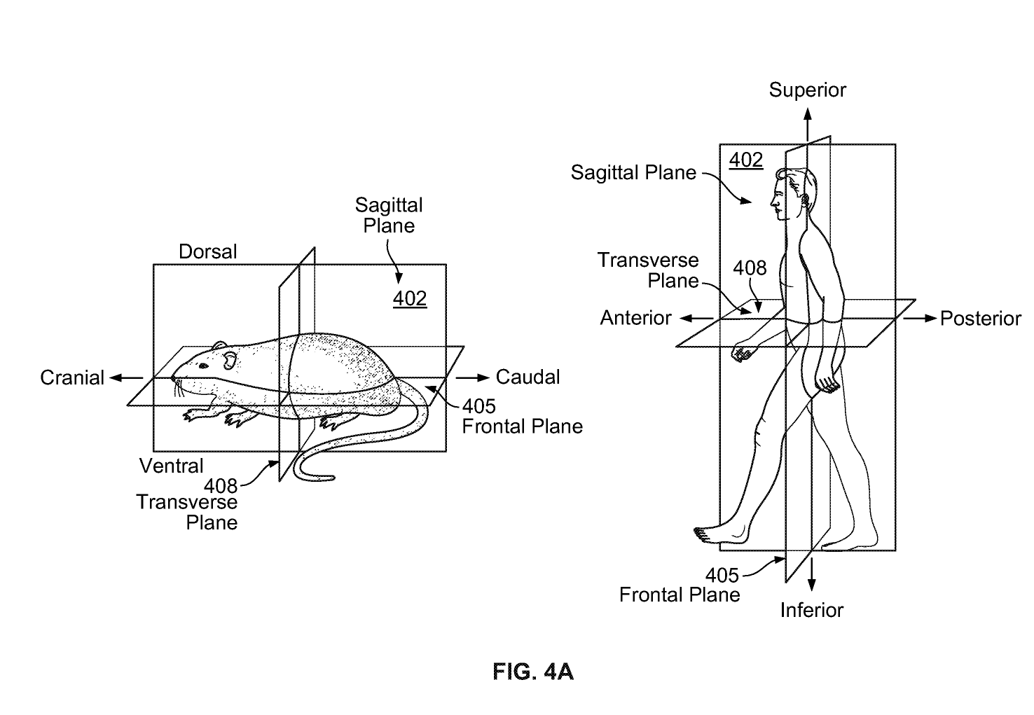

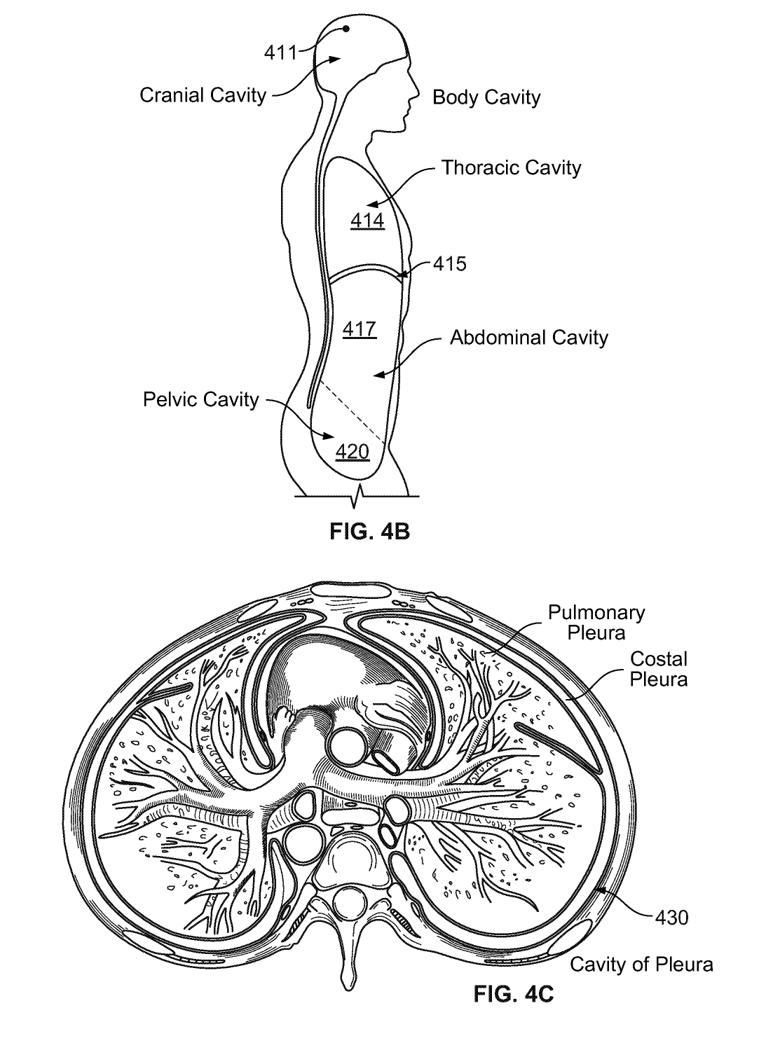

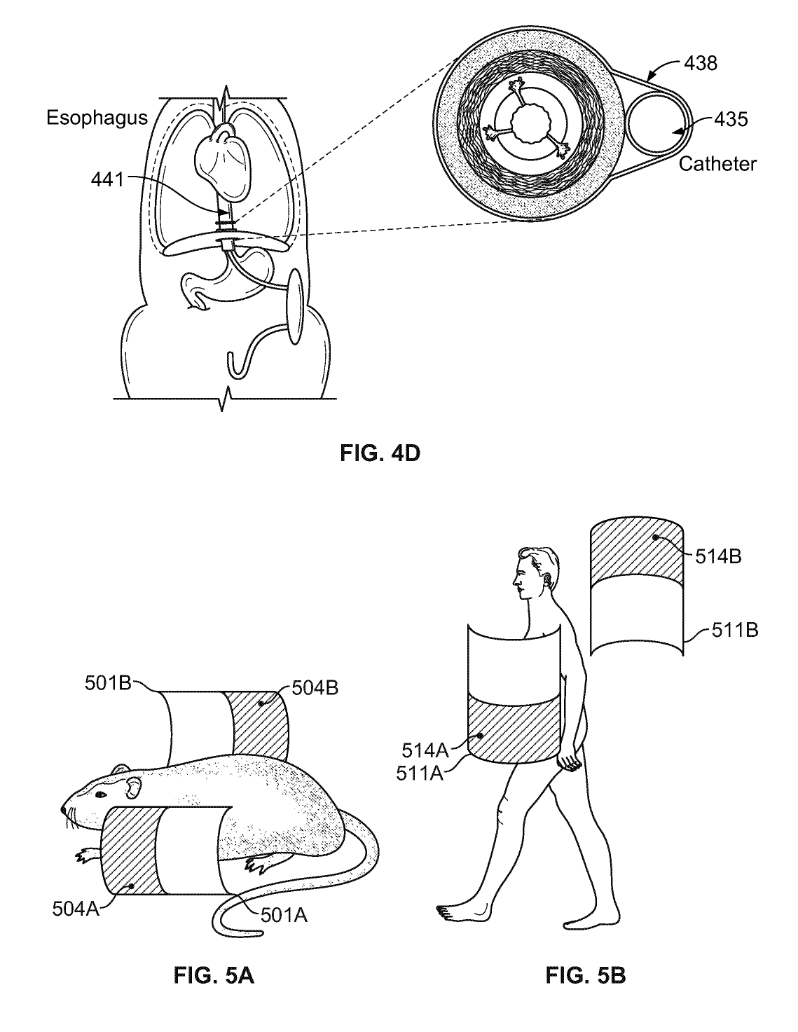

FIG. 4A is provided as a reference anatomical illustration to facilitate a more precise discussion of placement of various components of the implantable monitoring device 108 with reference to other figures. In particular, FIG. 4A illustrates various reference planes that are used to describe anatomy of either a four-legged animal (a quadruped), such as, for example, a laboratory rat; or a two-legged animal (biped), such as, for example, a human patient. As shown, a sagittal plane 402 bisects both quadrupeds and bipeds longitudinally into symmetrical left and right halves. A frontal plane 405 divides quadrupeds longitudinally into a front (ventral) portion and a back (dorsal) portion, and the frontal plane 405 divides bipeds longitudinally into a front (anterior) portion and a back (posterior) portion. A transverse plane 408 divides a quadruped between the head-end (cranial direction) and the tail-end (caudal direction) in a manner that is perpendicular to the sagittal and frontal planes (402 and 405, respectively), and a transverse plane 408 divides a biped between the head (superior direction) and the feet (inferior direction). Descriptions of FIGS. 4B, 4C and 4D are provided below, in context.

In light of the anatomical reference provided by FIG. 4A, sensor placement for capturing a high-quality thoracic impedance value is discussed in more detail with reference to FIGS. 5A and 5B. In general, and in lay terms, lead wires are placed in some implementations such that a line drawn between electrode(s) disposed on the two leads passes through at least a portion of the test subject's lungs.

FIG. 5A illustrates a laboratory rat surrounded by two halves 501A and 501B of a cylinder that is divided by the sagittal plane 402 (see FIG. 4A). Together, the two halves 501A and 501B depict a circumferential surface of the rat in the general chest and back region. In some implementations, two lead wires 270A and 270B (see FIG. 2D) are placed on the cylinder halves 501A and 501B in such a manner that a line connecting electrodes on the lead wires 270A and 270B crosses the test subject in either a lateral (side-to-side) manner, or front-to-back (ventral-to-dorsal) manner. In either case, the lead wires are generally placed (although they need not be) such that a line connecting the two leads crosses the sagittal plane 402.

In some implementations, the lead wires are further placed such that the line connecting them crosses the test subject at an angle relative to the transverse plane (such that one lead is more cranially disposed and one lead is more caudally disposed, resulting in a signal between the two leads crossing the sagittal plane 402 in a cranial-to-caudal manner). One possible way for signals between the leads (e.g., lead wires 270A and 270) to cross the sagittal plane in a cranial-to-caudal manner is for one lead wire (e.g., lead wire 270A) to be disposed subcutaneously in the general shaded region 504A and for the other lead wire (e.g., lead wire 270B) to be disposed subcutaneously in the general shaded region 504B. In general, regardless of the precise disposition of lead wires, the lead wires are disposed in some implementations such that signals between the lead wires pass through at least a portion of one of the test subject's lungs.

FIG. 5B illustrates similar detail for a biped (e.g., a human patient) as that shown in FIG. 5A for a quadruped (e.g., a laboratory rat). In particular, the biped is surrounded by two halves 511A and 511B of a cylinder that is divided by the sagittal plane 402 (see FIG. 4A). Together, the two halves 511A and 511B depict a circumferential surface of the biped in the general chest and back region. In some implementations, two lead wires 270A and 270B (see FIG. 2D) are placed on the cylinder halves 501A and 501B in such a manner that a line connecting electrodes on the lead wires 270A and 270B crosses the biped in either a lateral (side-to-side) manner, or front-to-back (anterior-to-posterior) manner. In either case, the lead wires are generally placed (although they need not be) such that a line connecting the two leads crosses the sagittal plane 402.

In some implementations, the lead wires are further placed such that the line connecting them crosses the test subject at an angle relative to the transverse plane (such that one lead is disposed more superior and one lead is dispose more inferior, resulting in a signal between the two leads crossing the sagittal plane 402 in a superior-to-inferior manner). One possible way for signals between the leads (e.g., lead wires 270A and 270) to cross the sagittal plane in a superior-to-inferior manner is for one lead wire (e.g., lead wire 270A) to be disposed subcutaneously in the general shaded region 514A and for the other lead wire (e.g., lead wire 270B) to be disposed subcutaneously in the general shaded region 514B. As in the case of the quadruped shown in FIG. 5A, lead wires are advantageously disposed in bipeds, in some implementations, such that signals between the lead wires pass through at least a portion of one of the test subject's lungs.

Regarding the depth of implantation, the lead wires may be implanted subcutaneously. For example, in some implementations, the electrodes are implanted just below the surface of the skin; in other implementations, the electrodes are implanted in or below muscle tissue; in yet other implementations, the electrodes are implanted more deeply in the test subject (e.g., in an internal cavity or organ).

In some implementations, it is advantageous to implant both sensor electrodes and other components of the implantable monitoring device (e.g., amplifiers 232-244, signal combiner 223, A/D circuitry 226 and transmitter 217) subcutaneously but external to the cranial, thoracic, abdominal or pelvic cavities. For reference, FIG. 4B illustrates these four cavities in a biped. The cranial cavity 411 generally refers to the space inside the skull, in which the brain is disposed, and also extends into the spine, where the brain stem and upper spinal cord are disposed. The thoracic cavity 414 generally refers to the space in which the lungs and heart are disposed, and may be considered to be bounded by the diaphragm 415. Boundaries between the abdominal cavity 417 and pelvic cavity 420 may be less precisely defined, but in general, the abdominal cavity 417 refers to the internal region below the thoracic cavity 414 and above the bones of the pelvis. The abdominal cavity 417 includes most of the internal organs (e.g., stomach, liver, gall bladder, spleen, pancreas, small intestine and large intestine). The pelvic cavity 420 is bounded by the bones of the pelvis and primarily contains reproductive organs and the rectum.

Primary advantages of implanting electrodes and other components of the implantable monitoring device outside of the above-described internal cavities can include a reduced risk of infection and fewer regulatory controls. With respect to regulatory controls, subcutaneous implantation may not be considered major surgery, whereas deeper implantation may be characterized as major surgery. In some cases, animal care regulations do not allow animals to undergo more than one major surgery. In such cases, the implantable device 108 can be subcutaneously implanted without invoking a major surgery limitation.

With respect to infection, it is generally understood that the deeper and more invasive a surgical procedure is, the greater the risks are of infection. That is, the risk of infection associated with a subcutaneous procedure where walls of the cranial cavity 411, the thoracic cavity 414, the abdominal cavity 417 or the pelvic cavity 420 are not pierced is generally understood to be lower than the risk of infection of a procedure in which surgical tools or implantable devices are introduced into any one of these cavities.

Minimizing risk of infection can be important to particular kinds of testing and research. For example, in the context of pharmaceutical development, technicians and scientists may be reluctant to deeply implant monitoring devices (e.g., implant monitoring devices in one of the cranial, thoracic, abdominal or pelvic cavities) for physiological monitoring aimed at assessing toxicity of a compound. In part, this may be related to a concern that infection, should it occur, may be more likely to skew test results related to toxicity testing than to skew test results related to, for example, other pharmacology testing.

The methods and devices described herein can provide significant advantages in the context of toxicity or safety pharmacology testing by providing a means for accurately monitoring physiological parameters of an ambulatory (e.g., unrestrained within the confines of a traditional test-subject housing), non-anesthetized animal, with less risk of infection than other methods involving more deeply implanted monitoring devices. In addition, as described in greater detail below, the devices and method described herein can be employed to capture a wide range of physiological data.

A description of data that can be captured by the device 108 that is implanted in the manner described above, and the processing of that data, is now provided. Table 1, below, depicts an example set of data that can be received from an implantable monitoring device.

TABLE 1
Sample data received
from the implantable monitoring device

| Time | Value (e.g., ohms) |
|---|---|
| . . . | . . . |
| 4526.000 | 67.749023 |
| 4526.005 | 67.718506 |
| 4526.010 | 67.718506 |
| 4526.015 | 67.718506 |
| 4526.020 | 67.718506 |
| 4526.025 | 67.657471 |
| . . . | . . . |

As depicted in Table 1, the data provided by the implantable device 108 (e.g., to an external system, such as the system 114) can include a time ordered series of physiological data points. In particular, pairs of values including a time reference and a corresponding physiological parameter can be provided in sequence (e.g., with reference to FIG. 2A, from the transmitter 217, in conjunction with the sensors 202-214, amplifiers 232-244, combiner 223 and A/D 226). In the example, of Table 1, actual impedance values are depicted, but in other implementations, raw data (e.g., voltage values) could alternatively be provided, and the raw data could be appropriately filtered and processed. For example, the voltage (e.g., voltage sensed by the voltage amplifier 259) could be demodulated based on a magnitude of an applied current (e.g., the current applied by the current generator 251) to determine instantaneous impedance values. The magnitude of the applied current could be provided in the data, or the magnitude of the applied current could be fixed, and the external system could be configured to determine impedance based on the fixed magnitude of the current.

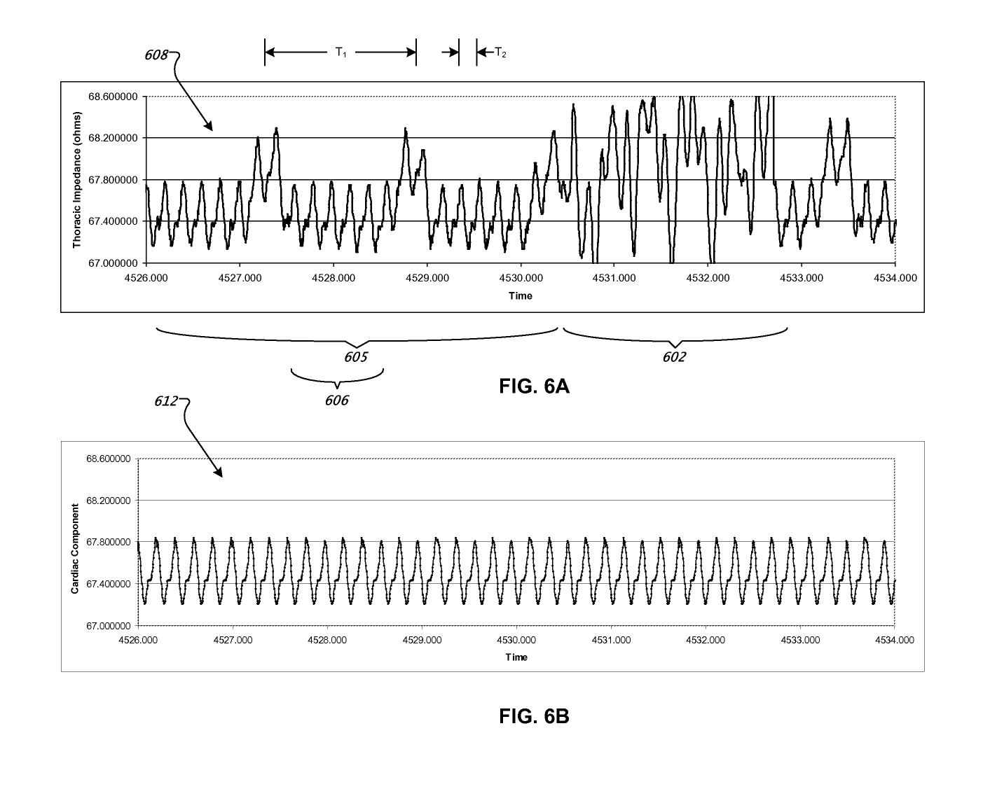

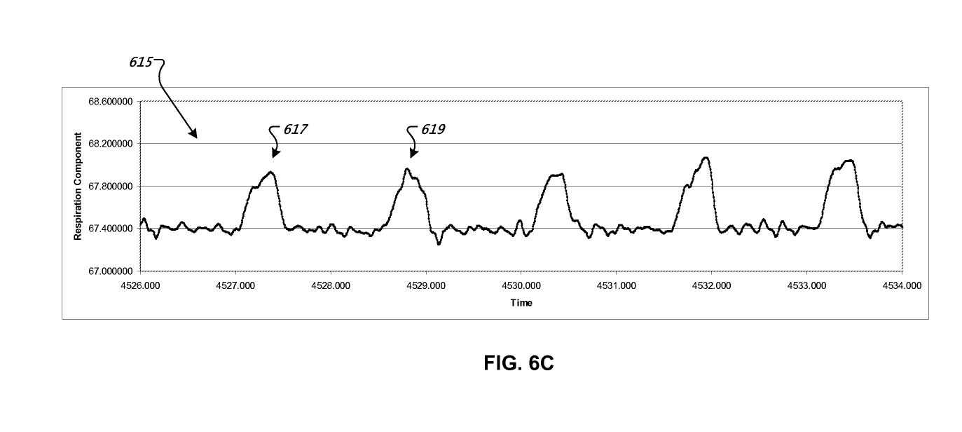

FIG. 6A graphically depicts data that can be received from the implantable monitoring device 108 (e.g., data of the form shown in Table 1). As shown, the data includes time-varying values of thoracic impedance. In some cases, quality of some portions of the data is lower than quality of other portions. For example, a region 602 depicts data of lower quality that may correspond to noise picked up by the electrodes (e.g., noise related to certain movements of the test subject or other interference or data corruption). In other regions (e.g., a region 605), quality of the data is much higher, and data in such regions can be extracted and analyzed.

In some implementations, a first step in processing data, such as that depicted in FIG. 6A, is identifying windows of data having clean, uncorrupted signals (e.g., windows such as the window 605). Identifying windows of data having clean, uncorrupted signals can, in some implementations, involve identifying corrupted data or data that is otherwise not likely to be useful. This can be done in various ways. For example, spectral analysis of the data can identify regions having content at a large number of frequencies, which may imply that the data has been corrupted (e.g., by the test subject's posture, the test subject's activity, or by some other source, such as radio interference with the transmitted signal). As another example statistical analysis of the data can identify regions having content that is statistically similar to patterns of known corrupted data. Once identified, such data can be removed, or alternatively, other data can be selected for further processing.

In some implementations, data can be removed through application of various kinds of filters. For example, digital or analog filters can be applied to remove undesirable data. The filter can be, for example, a linear, non-linear, histogram-based, or any other appropriate type of filter. Alternatively, other forms of signal processing (e.g., forms of signal processing that are not traditionally characterized as filtering) can be applied to the data to remove particular portions or qualities of the data.

As another example of identifying windows of data having clean, uncorrupted signals, the implanted system can include an accelerometer sensor to detect both posture and behavior or activity levels of the test subject. Data from the accelerometer sensor can be used to remove windows of impedance data corresponding to posture or activity of the test subject that may be likely to cause corruption of impedance data. As a particular example, impedance data may be corrupted by vigorous activity of the test subject (e.g., running); an accelerometer can be employed to detect such vigorous activity, and based on detection by the accelerometer of the vigorous activity, corresponding impedance data can be removed—either internal to the implanted device, before the data is sent, or external to the implanted device, in the external system.

As another example, the implanted system can include an electromyogram (EMG) sensor that can be employed to detect specific movements that may have a tendency to corrupt impedance data (e.g., certain movements of the front legs, in the case of a quadruped). In a similar manner as described above, impedance data that corresponds to EMG sensor-detected movements that are likely to corrupt the impedance data can be removed.

Once appropriate windows of data have been identified, the data can be processed to extract respiration-significant information. A brief qualitative description of typical impedance data is now provided as background to a discussion of extracting respiration-significant information. As shown in one example, the data 608 can appear as the superposition of a number of different impedance signals. In particular, the data can include a base impedance value (e.g., a DC value), an impedance signal having a first dominant frequency (e.g., a primary (non-harmonic) frequency of 1/T₁), and an impedance signal having a second, higher dominant frequency (e.g., a primary (non-harmonic) frequency of 1/T₂). The base impedance value can correspond to an impedance of the test subject that is primarily associated with bone, organs, and connective tissue, and may be influenced by how “wet” the test subject is internally.

In some implementations, the base impedance value changes with the test subject's mass. Moreover, base thoracic impedance can decrease based on fluid in the heart, lungs or elsewhere (e.g., pulmonary, thoracic or systemic edema), and base thoracic impedance can increase because of scarring or otherwise hardening of pulmonary tissue (e.g., as can happen in Chronic Obstructive Pulmonary Disease (COPD)).

The signal having the first frequency may generally correspond to changes in impedance associated with respiration (e.g., changes in impedance caused by changes in volume of highly resistive air in the lungs of the test subject). The signal having the second frequency may generally correspond to changes in impedance associated with cardiac function (e.g., changes in impedance caused by changes in volume of highly conductive blood in the heart of the test subject).

To identify the signal having the first frequency (e.g., the signal associated with respiration parameters), some implementations identify and remove from the data 608 the signal having the second frequency (e.g., the signal associated with cardiac function). The signal having the second frequency can be identified in a number of ways. In some implementations, spectral analysis is performed on the data 608, and the frequency content is identified. A filter can then be constructed to remove periodic signals having higher frequency content (or other forms of signal processing can be applied to achieve the same result, as indicated above).

In some implementations, the implanted device employs additional sensors to help identify cardiac signals. For example, some implanted devices employ electrodes (e.g., separate electrodes, or the electrodes used to apply the current signal or detect the corresponding voltage signal, for purposes of obtaining impedance values) to obtain electrocardiogram (ECG) data from the test subject, then remove ECG data from the signal 608 to obtain data associated with respiration. As another example, some implanted devices employ a pressure sensor to measure blood pressure, then use data associated with blood pressure measurements to remove a cardiac-related signal from the data 608. The above description provides a few examples of identifying respiration information in the data 608, but the reader will appreciate that any suitable filtering or signal processing technique can be employed. FIG. 6B graphically depicts a cardiac signal 612 that has been identified in the data 608.

FIG. 6C graphically depicts data 615 after the signal having the second frequency has been removed (e.g., the cardiac signal 612). In particular, FIG. 6C illustrates a signal 615 that corresponds to a change in thoracic impedance associated with respiration function. More specifically, the signal 615 is relatively flat (corresponding to a relatively constant respiration component of thoracic impedance) when the test subject is not actively inhaling or exhaling. When the test subject inhales and exhales, the respiratory component of thoracic impedance peaks (e.g., at points 617 and 619), due to the increased resistance of the greater volume of air in the lungs during inspiration and until the inspired air is exhaled.

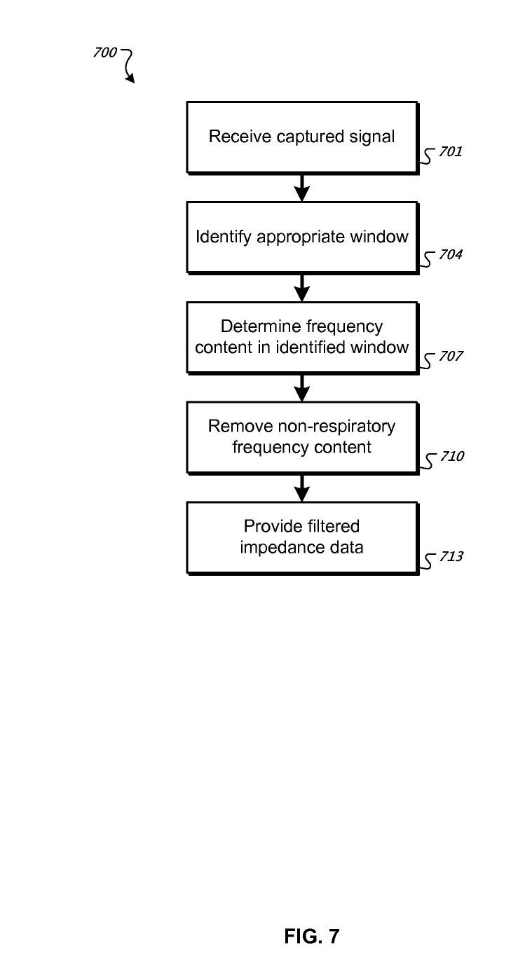

FIG. 7 is a flow diagram of an example method 700 for obtaining respiration-based thoracic impedance data. The method 700 can include receiving (701) a captured signal that is representative of thoracic impedance. For example, with reference to FIGS. 1, 2A and 2B, a device 108 implanted in a test subject can generate a current signal (using a current generator 251) between two electrodes 253A and 253B and can detect a corresponding voltage between two electrodes 256A and 256B that is modulated by an impedance (e.g., a thoracic impedance) between the electrodes 256A and 256B. The voltage signal can be demodulated to form a time-varying impedance signal. In some implementations, the voltage signal can be demodulated within the implantable device 108, and a time-ordered sequence of impedance values can be transmitted to the external system 114 (e.g., with the transmitter 217). In other implementations, the voltage signal can be directly transmitted (e.g., with the transmitter 217), and the voltage signal can be demodulated in the external system 114 (e.g., based on a transmitted time-ordered sequence of magnitude values corresponding to the current signal, or based on a fixed magnitude of current stored in the external system 114). In graphical form, the captured signal can appear in the form of the signal 608, shown in FIG. 6A.

The method 700 can include identifying (704) an appropriate window of data in the captured signal. In particular, for example, the method 700 can include identifying and removing (e.g., filtering out) data that is likely to be corrupted (e.g., based on posture or movement of the test subject, or based on other interference). Such corrupted data is depicted in region 602, shown in FIG. 6A, and may be identified, in some implementations, by spectral analysis of the signal 608. Whereas data that has not been corrupted may only have frequency content at a small number of primary frequencies (e.g., two), data that has been corrupted may have frequency content at many more primary frequencies. In some implementations, windows of data that are likely to be corrupted, or otherwise not usable for further analysis, can be identified through the use of other sensors, such an accelerometer or an EMG sensor. Appropriate windows of data can include data that is not identified as likely to be corrupted. For example, with reference to FIG. 6A, a region 605 may be identified as an appropriate window of data for further analysis.

Identification (704) of an appropriate window of data can take place in the implantable device 108 or external to the implanted device. For example, in some implementations, the implanted device includes an accelerometer, and any time data from the accelerometer indicates a high rate of activity of the test subject that may corrupt impedance data, the impedance data (or corresponding voltage signal data) is not sent; at other times, when accelerometer data indicates a lower rate of activity, the impedance data (or corresponding voltage signal data) is sent to the external system. In other implementations, data analysis software in the external system 114 can be employed to identify (704) appropriate windows of data for further analysis.

The method 700 can include determining (707) frequency content in the identified window of data. In many implementations, data in the identified window will have two primary frequencies—one frequency associated with cardiac function, and a second, generally lower frequency associated with respiration function. Determining (707) frequency content can include identifying these two primary frequencies. The determination can be made in various ways, including through spectral analysis of the data. The frequencies can be identified by processing elements (not shown in the figures) that are internal to the implantable device 108, or by the external system 114.

The method 700 can include removing (710) non-respiratory frequency content in the identified window of data (e.g., by filtering the captured signal). Removing (710) non-respiratory frequency content can include configuring a filter to remove a cardiac signal, such as the signal 612 that is shown in FIG. 6B. The higher of frequencies determined (707) above can be used to configure a frequency of the filter, and an amplitude associated with the signal 608 during a period of time 606 (see FIG. 6A) that corresponds primarily to cardiac function can be used to configure an amplitude for the filter. In some implementations, the period of time 606 is identified based on identification of the relatively uniform amplitude of the thoracic signal 608 during this period. FIG. 6C illustrates one example set of data 615 post-filtering.

The method 700 can include providing (713) filtered impedance data. This data can be provided (713) to the external system 114 by the implanted device in implementations where the data is processed internal to the implantable device 108. In implementations in which the data is processed (e.g., filtered) in the external system 114, the filtered thoracic impedance data can be provided (713) to other analysis tools in the external system 114.

In some implementations, a linear correlation has been established between the change in thoracic impedance during respiration and the volume of air inhaled and exhaled (e.g., the tidal volume). Thus, with appropriate calibration, the data 615 shown in FIG. 6C can be employed to determine various respiration parameters. In particular, for example, time between peaks of the respiration component of thoracic impedance can be measured to determine respiration rate. Each peak itself (once calibrated) can be used to determine a corresponding tidal volume. The slope of the change in the respiratory component of thoracic impedance during a respiration cycle can be used to determine an inspiratory flow, an expiratory flow, and inspiratory time or interval, or an expiratory time or interval. Such a slope can be determined by calculating the derivative for each point along the thoracic impedance curve. Other parameters can also be determined using the respiratory component of thoracic impedance or its slope. In particular, for example, with a pressure sensor appropriately disposed in the test subject an airway resistance or lung compliance can also be determined. As another example, thoracic edema can be determined.

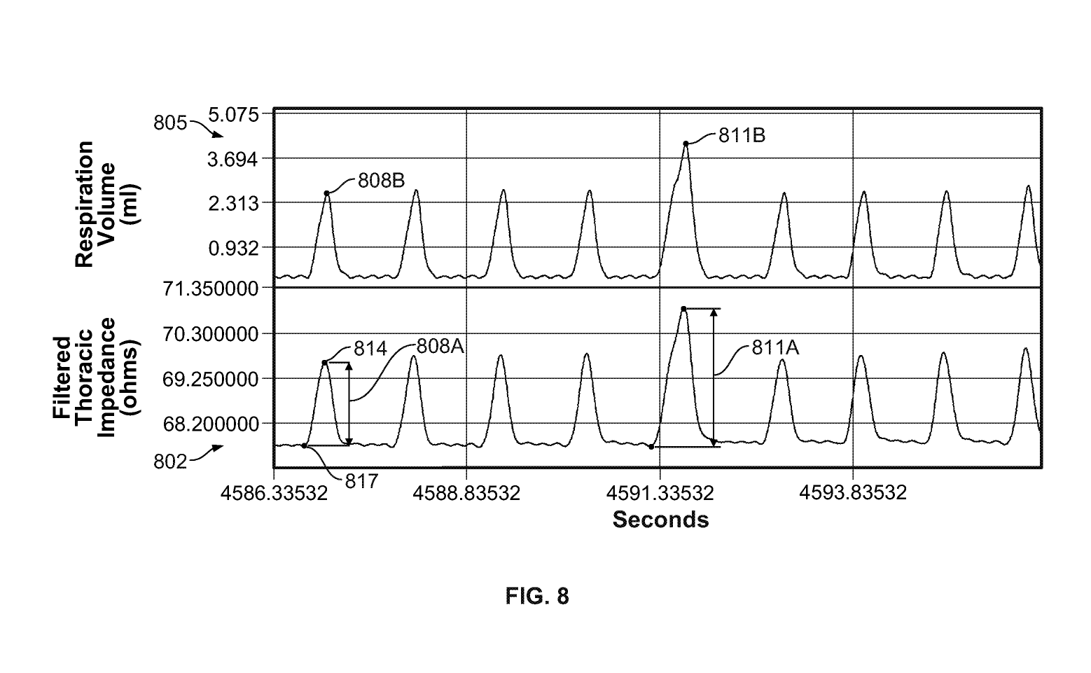

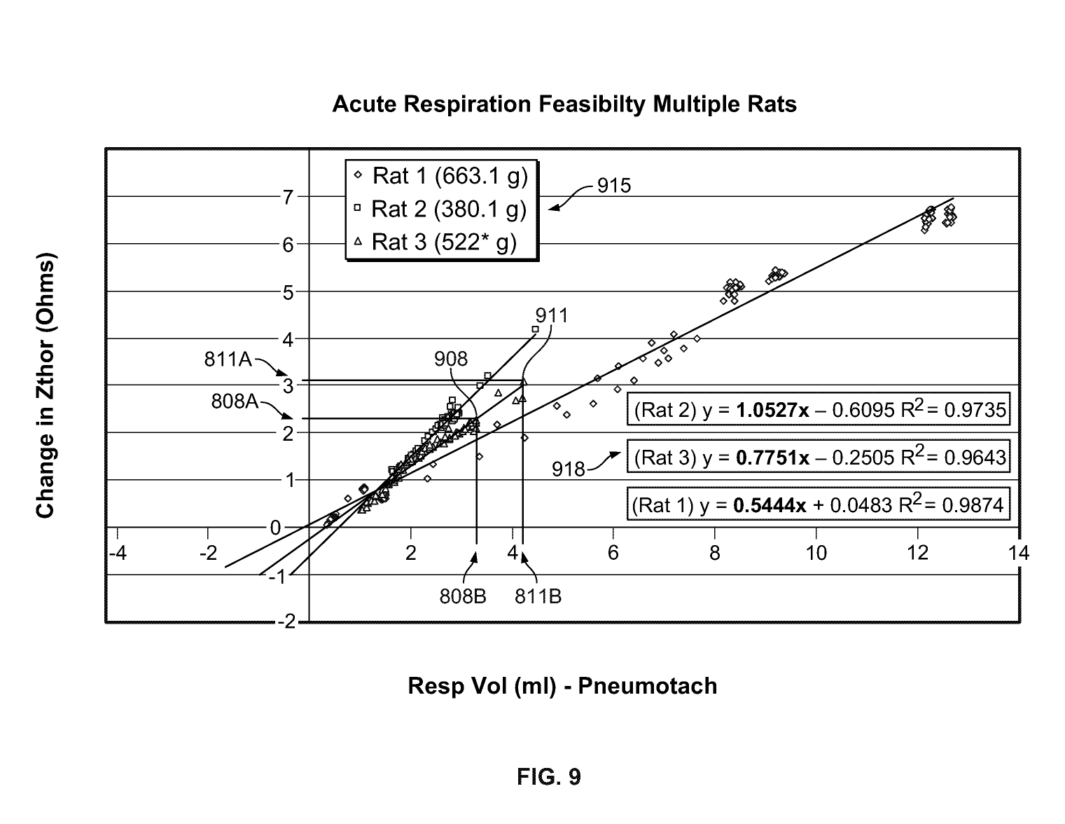

Calibration of the respiratory component of thoracic impedance is now described with reference to FIGS. 8 and 9. In some implementations, thoracic impedance data for a particular test subject is calibrated based on a separate measurement of respiration parameters (e.g., tidal volume) using equipment other than the implantable device 108. In particular, some implementations employ a pneumatach, or some other kind of plethysmography chamber to obtain respiration data from a test subject.

FIG. 8 illustrates example data 802 corresponding to thoracic impedance obtained from the implantable device. As shown, the data 802 is more filtered than the data 615 shown in FIG. 6C. FIG. 8 further illustrates corresponding respiration data 805 (e.g., respiratory volume vs. time) 805, which, in this example, was obtained from the test subject with an external device (a pneumatach) at the same time the thoracic impedance data 802 was obtained. In some implementations, obtaining the respiration data 805 from the test subject with a pneumatach or other external device (e.g., a plethysmography chamber) requires the test subject to be substantially restrained (e.g., afforded little, if any, movement relative to that afforded by a standard housing, in which the test subject is confined to a reasonably sized area suitable for long-term housing, but allowed to freely move within that area). Note that respiration volume is illustrated in FIG. 8, but respiration flow may have been obtained from the external device. The reader will appreciate that in such a case, respiration volume can be obtained by integrating the respiration flow.

The data 802 and 805 can be used to calibrate the thoracic impedance data 802 to actual tidal volume—for example, to facilitate determination of tidal volume and other parameters that can be derived from tidal volume, when the test subject is not restrained by an external device, such as a pneumatach. Calibrating the impedance data 802 can include calibrating a linear relationship between thoracic impedance and actual respiratory flow or volume—that is, determining constants of a linear equation that relates thoracic impedance to tidal volume for a particular test subject.

Qualitatively, there is direct correlation between the overall change in thoracic impedance (that is, the respiratory component of thoracic impedance, which is implied in the discussion that follows) during a respiration cycle and the tidal volume associated with that respiration cycle. FIG. 8 graphically depicts these values as follows: a change in impedance 808A for a first respiratory cycle corresponds to measured tidal volume 808B (peak respiration volume); similarly, a change in impedance 811A for a second respiratory cycle corresponds to a measured tidal volume 811B. In some implementations, an overall change in thoracic impedance can be determined by identifying a maximum thoracic impedance for a given respiration cycle (e.g., the maximum thoracic impedance 814 in the first respiration cycle) and subtracting from it a minimum thoracic impedance for the same respiration cycle (e.g., the minimum thoracic impedance 817 in the first respiration cycle). By identifying a number of changes in thoracic impedance and corresponding measured tidal volumes, constants in the linear equation can be identified, and the linear equation with the identified constants can be employed with other thoracic impedance data to calculate a corresponding tidal volume.

Additional details related to identifying (solving for) the constants in the linear equation are described with reference to FIG. 9. FIG. 9 illustrates a plot of various data points that represent corresponding pairs, where each data pair includes a change in thoracic impedance and a corresponding respiration volume. Respiration volume is plotted along the x-axis, and change in thoracic impedance is plotted along the y-axis. With reference to FIGS. 8 and 9, a data point 908 corresponds to the change in thoracic impedance 808A and the corresponding respiration volume 808B; a data point 911 corresponds to the change in thoracic impedance 811A and the corresponding respiration volume 811B. For purposes of example, many other data points are plotted, both for the same test subject 915 (“Rat 3”) and for two other test subjects (“Rat 1” and “Rat 2”).

By analyzing various data points for a particular test subject, a linear equation can be fit to the data (e.g., using a linear regression method) in order to determine a slope and offset for the equation. In the example shown in FIG. 9, the equation 918 for the test subject 915 has been determined to be:

y(Δohms)=0.7751(ohms/ml)*x(ml)−0.2505(ohms)

That is, change in thoracic impedance, in ohms, for test subject 915, is substantially equal to 0.7751 times the tidal volume, in mL, minus 0.2505 ohms. The reader will appreciate that this equation can be rearranged to calculate tidal volume, in mL, based on a corresponding change in thoracic impedance (e.g., as determined by the implantable device 108). In particular:

$$x ( ml ) = \frac{y ( Δ ohms ) + 0.2505 ( ohms )}{0.7751 ( ohms \text{/} ml )}$$

Data for other test subjects is also shown in FIG. 9. As shown, the slope of change-in-impedance/tidal volume decreases based on the mass of the test subject. Accordingly, without higher-order calculations, the calibration relationship for a particular test subject may only be accurate for a period of time during which the mass of the test subject remains relatively constant. In some implementations, certain test subjects within a particular species may have a uniform enough impedance change/tidal volume/mass relationship that an equation can be solved relating all three parameters. For example, thoracic impedance and/or respiration volume data may be normalized based on animal mass, and the a linear regression technique may be applied to normalized data taken from a large enough sample of homogenous test subjects (e.g., the same or similar species, same general age, same general condition of health, etc.) to determine a unified equation relating tidal volume to both change in thoracic impedance and mass of the test subject. In other implementations, the calibration process described with reference to FIGS. 8 and 9 is an acute calibration process for use with a single test subject and may need to be periodically repeated.

To characterize how well each linear equation describes the relationship between actual change-in-thoracic-impedance values and corresponding tidal-volume values for a given test subject, linear regression of the data depicted in FIG. 9 has been performed for each test subject, and the R² value is shown. For the test subject 915 (“Rat 3”), the R² value is shown to be 0.9643—indicating a very good fit between the data and the above-described corresponding linear equation. Linear equations and corresponding R² values for other test subjects (“Rat 1” and “Rat 2”) are also shown.

The data points are plotted to illustrate graphically the linear relationship between change in impedance and corresponding respiratory volume. The reader will appreciate, however, that the relationship between change in thoracic impedance and corresponding tidal volume can be determined without actually plotting the data. For example, in many implementations, the relationship is calibrated (that is, the constants in the equation are determined), by numerically analyzing various data points from thoracic impedance data captured from the test subject.

FIG. 9 illustrates a process to fit a linear equation to impedance data. The principles described herein can also be applied to fit a non-linear equation to impedance data. Non-linear fitting may be advantageous in certain animals, or in animals having certain conditions. For example, non-linear fitting of data may be particular applicable to the development of disease models, particularly, for example disease models that track changes to the respiratory system of test subjects as a disease progresses. Other applications of non-linear fitting are contemplated.

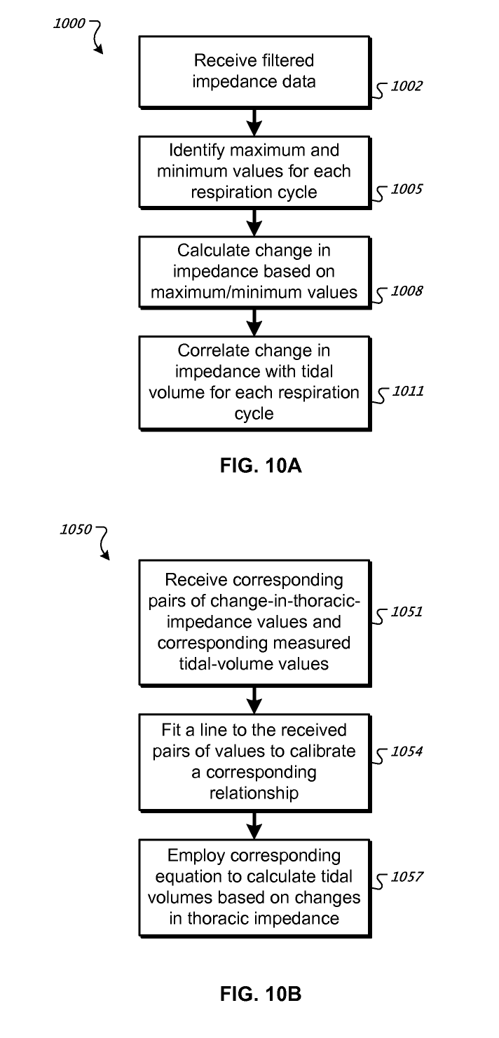

FIG. 10A is a flow diagram of an example method 1000 for determining a tidal volume of a test subject based on a calibrated relationship between change in thoracic impedance and corresponding tidal volume. The method 1000 can include receiving (1002) filtered impedance data. In particular, for example, a processing unit (e.g., the external system 114 or a processor (not shown) in the implantable device 108) can receive filtered thoracic impedance data, such as the data 615 shown in FIG. 6C or the data 802 shown in FIG. 8.

The method 1000 can include identifying maximum and minimum values for each respiration cycle. In particular, with reference to FIG. 8, the method 1000 can include determining a maximum and minimum value for each respiration cycle, such as the minimum value 817 and the maximum value 814.

The method 1000 can include calculating a change in impedance for each respiration cycle based on corresponding maximum and minimum thoracic impedance values. In particular, for example, the method 1000 can include calculating the difference between the maximum and minimum values 814 and 817 to determine an overall change in thoracic impedance 808A for the corresponding respiration cycle.

The method 1000 can include correlating the change in impedance with a tidal volume for each respiration cycle, based on a calibrated relationship between tidal volume and change in impedance for the test subject from which the impedance data was gathered. In particular, for example, the method 1000 can transform a change in thoracic impedance to a tidal volume, based on the above-described calibrated relationship (e.g., equation 918).

The method 1000 can be performed in either the implanted device (e.g., calibration parameters can be stored in memory in the device (memory not shown in the figures) and accessed by a processing unit (not shown in the figures) to calculate corresponding tidal volume. In other implementations, the method 1000 is performed outside of the implantable device 108 (e.g., in the external system 114).

FIG. 10B a flow diagram of an example method 1050 for calibrating a relationship between tidal volume and change in thoracic impedance. The method 1050 can include receiving (1051) corresponding pairs of change-in-thoracic-impedance values and measured tidal-volume values. For example, with reference to FIG. 9 the method 1050 can include receiving pairs of values such as the pairs graphically depicted by points 908 and 911. Each pair of values can represent a different value for either or both of change in thoracic impedance and corresponding respiration volume. Preferably, a range of values for each parameter is included in the pairs of data.

Based on the received pairs of values, a line can be fit (1054) to the data (e.g., a best-fit linear equation can be fit to the data), using, for example, linear regression or any other appropriate algorithm. Based on the fitted line, the corresponding equation can be employed (1057) to calculate tidal volume based on measured changes in thoracic impedance (e.g., changes measured by a device, such as the implantable device 108).

In various implementations, additional processing of impedance data or tidal volume data is desirable. For example, it may be desirable to normalize a modulated impedance (e.g., the AC component in a time-varying impedance value) relative to a base impedance (e.g., the DC component in a time-varying impedance value). In particular, as indicated above, base thoracic impedance can decrease as a result of fluid accumulation in the heart or lungs, and base thoracic impedance can increase as a result of scarring of pulmonary tissue. Other factors can also affect base thoracic impedance (e.g., changing mass of the test subject). Because amplitudes of the modulated impedance are, in some implementations, proportional to the base impedance, effects on thoracic impedance data of changing base impedance can be reduced by normalizing the modulated impedance relative to the base impedance.

In some implementations, it may be desirable to normalize thoracic impedance in other ways. For example, thoracic impedance can be normalized based on body temperature of the test subject. As another example, thoracic impedance can be normalized or filtered based on body posture (e.g., as detected by an accelerometer sensor).

Other processing of tidal volume can result in determination of other respiration parameters. For example, calculating a derivative (or difference values) of a time-varying series of tidal volume values (e.g., a series of tidal volume values determined from a corresponding series of thoracic impedance values) can yield a time-varying series of respiration flows. Positive flows can correspond to an inspiratory phase of inspiration, and negative flows can correspond to an expiratory phase of inspiration. Based on these values, parameters such as peak inspiratory flow, peak expiratory flow, inspiratory time or interval and expiratory time or interval can readily be determined.

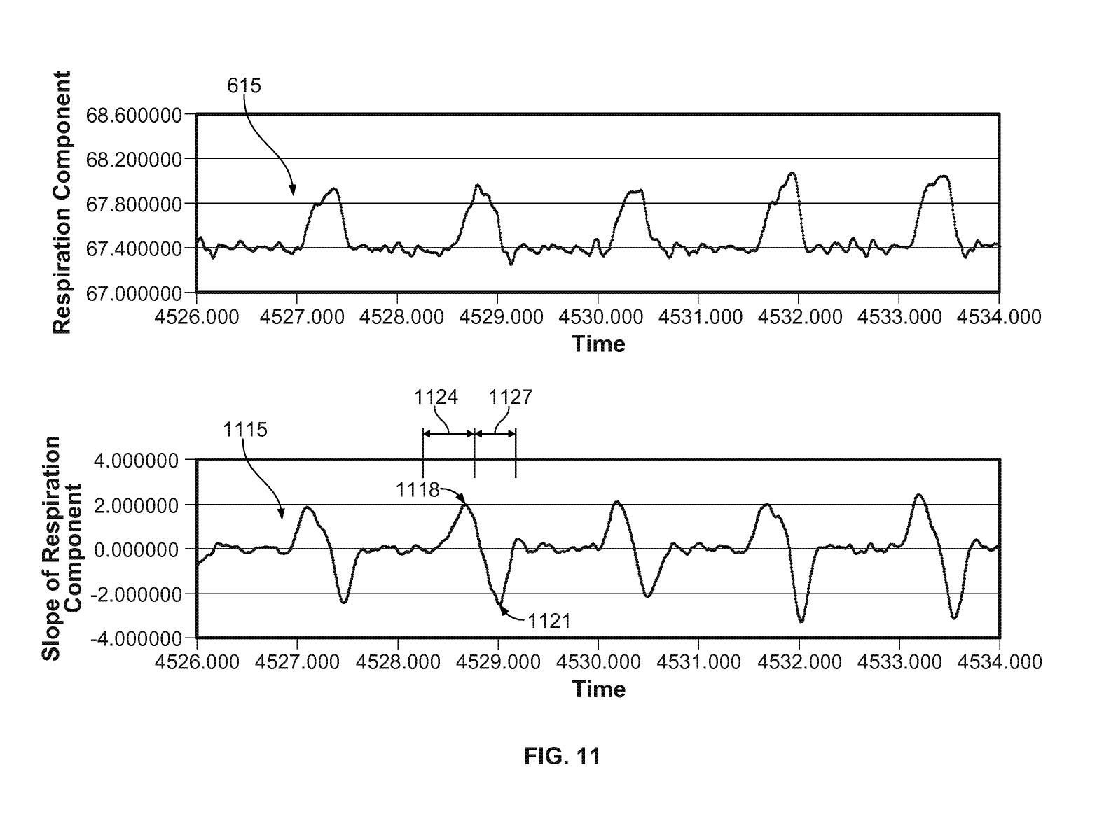

FIG. 11 illustrates a time-varying sequence of derivatives 1115 of a corresponding time-varying sequence of tidal volume values 615 (shown in FIG. 6C, but repeated in FIG. 11 for context). The sequence 1115 of derivative values graphically represents the slope of corresponding tidal volume values 615; conceptually, the derivative values represent a respiratory flow. Thus, a point 1118 represents a peak inspiratory flow for a particular respiration cycle, and point 1121 represents a peak expiratory flow for the respiration cycle. With appropriate calibration, actual flows can be derived from values the 1115. Time 1124 represents an inspiratory time, and time 1127 represents an expiratory time.

Additional respiratory parameters can be measured if the implantable device 108 includes additional sensors. For example, with an appropriately placed pressure sensor, pressure data can be combined with tidal volume data to obtain a measurement of lung compliance of a test subject.

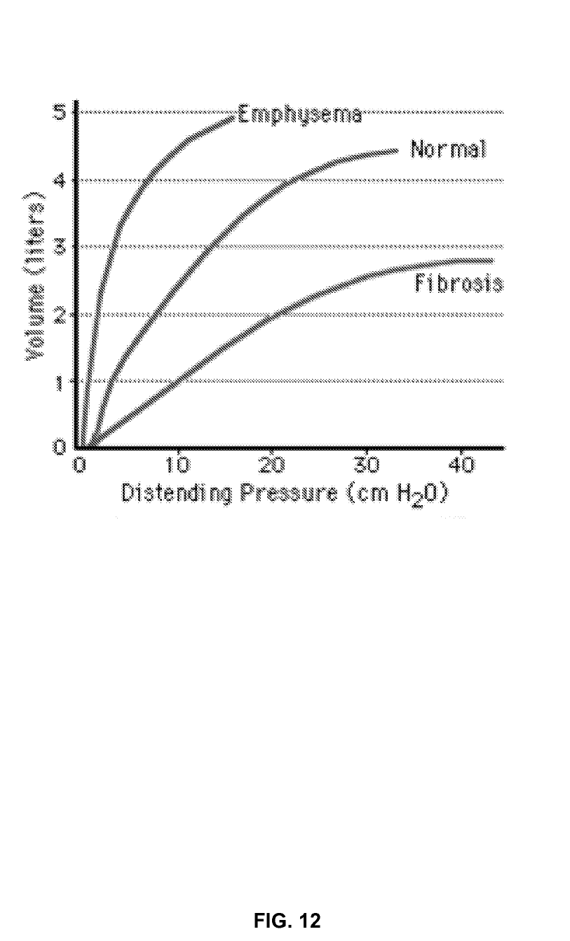

Lung compliance, or elasticity of the lungs, is generally determined by measuring transpulmonary pressure required to maximally inflate the lungs. Compliance can be affected by various conditions and diseases, such as accumulation of fibrous tissue in the lungs, or by edema in the alveolar spaces. Compliance can also be affected by age or pulmonary emphysema. To determine compliance of a test subject, pressure needed to distend the lungs of a test subject can be plotted or traced relative to tidal volume of the lungs. For illustration and reference, FIG. 12 shows three different compliance curves (volume vs. distending pressure) that illustrate a normal lung, a stiff lung (“fibrosis” curve) and a loose or elastic lung (“emphysema” curve).

A pressure measurement for calculating compliance as described above can be obtained in a number of ways. In some implementations, a pressure sensor can be inserted directly into the pleural space of the test subject. For reference, a cross-section of an example test subject's lungs is provided in FIG. 4C with an indication of the pleural space 430. In other implementations, a pressure sensor (e.g., a pressure catheter 435) can be inserted below the serosal layer 438 of the esophagus 441 of a test subject, as depicted in FIG. 4D.

The above-described pressure measurement can also be used in conjunction with respiratory airflow to determine airway resistance of a test subject. Airway resistance characterizes the opposition to flow caused by forces of friction and can be used to evaluate, for example, the bronchiorestrictive effect of various substances. Airway resistance can be determined by measuring the transpulmonary pressure required to generate maximal airflow during inspiration or expiration. Thus, airway resistance can be determined based on a pressure measurement from the pleural cavity or the esophagus of the test subject, in conjunction with a corresponding peak inspiratory flow or expiratory flow measurement.

A number of implementations have been described. Nevertheless, it will be understood that various modifications may be made without departing from the spirit and scope of the disclosed implementations. For example, various examples are provided in the context of testing or research of pharmaceutical compounds. However, the reader will appreciate that the systems and methods described herein can be employed to test respiratory effect (or other physiological effect) of any kind of substance, such as for example, substances related to bio-defense or bio-weaponry, substances associated with environmental concerns; or respiratory effect (or other physiological effect) of any kind of stimulus, such as neuron-stimulation. In addition, the systems and methods described herein can be employed to determine or study disease progression during animal model development, or for any other kind of basic research. The systems and methods can be employed in various types of living beings, for various purposes—including all types and sizes of laboratory animals (e.g., rodents, canines, non-human primates, pigs, etc.), other animals that may be used in basic research (e.g., horses, fish, birds, etc.), as well as in human patients. Filtering is discussed herein to process data in various manners; the reader should appreciated that filtering can include various forms of signal or data processing—including those forms that may not be strictly characterized as “filtering.” Tidal volumes are described in some contexts above as being determined based on a time-ordered series of impedance values. In some implementations, tidal volumes can also be determined based on a single peak impedance value, from which a base impedance value (e.g., an average base impedance value) can be subtracted. In other implementations, tidal volumes and other respiration parameters can be determined based on other non-time-ordered impedance values (e.g., a histogram of values). Determining inspiratory and expiratory parameters can include analyzing corresponding tidal volumes (e.g., calculating a derivative of respiration volume over time), as described above, but certain inspiratory and expiratory parameters can also be directly calculated. In particular, for example, inspiratory time or interval can be determined based on a time between a point at which impedance is at a base value and a time at which impedance is at a peak value; similarly, expiratory time can be determined based on a time between a point at which impedance is at a peak value and a time at which impedance is again at the base value. Accordingly, other implementations are within the scope of the following claims.

What is claimed is:

1. A method of determining a value for a respiration parameter in a test subject, the method comprising:
capturing—using a fully implanted system that is comprised of a wireless transmitter and at least a first lead wire having a first electrode disposed thereon and a second lead wire having a second electrode disposed thereon, wherein the at least first and second lead wires are positioned in the test subject subcutaneously and external of any cranial, thoracic, abdominal and pelvic cavities of the test subject—information indicative of an impedance measure between the first and second electrodes and across a thoracic region of the test subject;
wirelessly transmitting, from the implanted system and to external equipment, the captured information;
determining in the external equipment a value for a respiration parameter of the test subject based on the wirelessly transmitted information; and
using the determined respiration parameter in safety pharmacology testing or toxicity testing to assess impact of a pharmaceutical compound on the test subject's respiratory system.

2. A method of determining a value for a respiration parameter in a test subject, the method comprising:
capturing—using a fully implanted system that is comprised of a wireless transmitter and at least a first lead wire having a first electrode disposed thereon and a second lead wire having a second electrode disposed thereon, wherein the at least first and second lead wires are positioned in the test subject subcutaneously and external of any cranial, thoracic, abdominal and pelvic cavities of the test subject—information indicative of an impedance measure between the first and second electrodes and across a thoracic region of the test subject;
wirelessly transmitting, from the implanted system and to external equipment, the captured information; and
determining a respiration parameter of the test subject based on the captured information.

3. The method of claim 2, wherein the electrodes are positioned in the test subject such that the impedance measure between the electrodes is made across the thoracic region of the test subject in manner that crosses a sagittal plane of the test subject in a cranial-to-caudal or posterior-to-inferior manner.

4. The method of claim 2, wherein the electrodes are positioned in the test subject such that the impedance measure between the electrodes is made through at least a portion of one of the test subject's lungs.

5. The method of claim 2, wherein the transmitter is positioned subcutaneously and external of any cranial, thoracic, abdominal and pelvic cavities of the test subject.

6. The method of claim 2, wherein capturing the information indicative of the impedance measure comprises injecting a current between two electrodes and measuring a resulting voltage between two electrodes.

7. The method of claim 6, wherein the fully implanted system comprises four electrodes, and wherein the two electrodes between which current is injected and the two electrodes between which the resulting voltage is measured are each distinct electrodes.

8. The method of claim 6, wherein the captured information comprises a value for the measured resulting voltage.

9. The method of claim 2, wherein the test subject is selected from the group of laboratory animals consisting of rodents, bovines, canines, ovines, porcines and non-human primates.

10. The method of claim 2, wherein the test subject is a human patient.

11. The method of claim 2, wherein the impedance measure comprises a base impedance component and a modulated impedance component, and wherein determining the respiration parameter comprises normalizing the modulated impedance component relative to the base impedance component.

12. The method of claim 2, wherein determining the respiration parameter of the test subject based on the captured information comprises identifying in the captured information first periodic data having a first dominant frequency and second periodic data having a second dominant frequency that is lower than the first frequency, and determining the respiration parameter of the test subject based on the second periodic data.

13. The method of claim 12, wherein the fully implanted system comprises a pressure sensor configured to obtain blood pressure information from the test subject, and wherein identifying in the captured information the first periodic data comprises identifying the first periodic data based on the blood pressure information.

14. The method of claim 12, wherein the fully implanted system comprises a sensor configured to obtain electrocardiogram (ECG) data from the test subject, and wherein identifying in the captured information the first periodic data comprises identifying the first periodic data based on the ECG data.

15. The method of claim 12, further comprising:
identifying in the captured information third data having spectral content that indicates that the third data has been corrupted by at least one of the test subject's posture or the test subject's activity; and
removing the third data from the captured information.

16. The method of claim 15, wherein the fully implanted system comprises an accelerometer sensor, and wherein identifying in the captured information the third data comprises identifying the third data based on data from the accelerometer sensor.

17. The method of claim 15, wherein the fully implanted system comprises an electromyogram (EMG) sensor, and wherein identifying in the captured information the third data comprises identifying the third data based on data from the EMG sensor.

18. The method of claim 2, wherein capturing the information indicative of the impedance measure comprises capturing the information when the test subject is unrestrained and unanesthetized.

19. The method of claim 2, further comprising using the determined respiration parameter in safety pharmacology testing or toxicity testing to assess impact of a pharmaceutical compound on the test subject's respiratory system.

20. The method of claim 2, wherein determining the value for the respiration parameter of the test subject comprises determining a tidal volume, the method further comprising measuring tidal volume of the test subject with a device that is external to the test subject, and calibrating a relationship between the captured information and the measured tidal volume.

21. The method of claim 20, wherein determining the value for the tidal volume of the test subject comprises correlating the captured information with the tidal volume based on the calibrated relationship.

22. The method of claim 21, wherein the calibrated relationship is a function of mass of the test subject, the method further comprising normalizing the tidal volume based on a mass of the test subject.

23. The method of claim 2, wherein determining the value for the respiration parameter of the test subject comprises identifying a time-ordered sequence of impedance values in the captured information, and determining, based on the time-ordered sequence of impedance values, at least one of a tidal volume, an inspiratory time, an expiratory time, an inspiratory flow or an expiratory flow of the test subject.

24. The method of claim 2, wherein the fully implanted system further comprises a pressure sensor, the method further comprising capturing, with the pressure sensor, information indicative of an internal pressure of the test subject.

25. An implantable device configured to monitor thoracic impedance in a test subject, the implantable device comprising:
a fully implantable system that is comprised of controller and at least a first lead wire and a second lead wire, wherein the controller and the first and second lead wires are configured to be subcutaneously implanted in the test subject, external of any cranial, thoracic, abdominal or pelvic cavities of the test subject;
wherein, the first lead wire has first and second electrodes disposed thereon and the second lead wire has third and fourth electrodes disposed thereon; and
wherein the controller comprises a) a current signal generator that generates a current signal between the first and third electrodes; b) a voltage amplifier that detects a voltage difference between the second and fourth electrodes; and c) a wireless transmitter that is configured to transmit information to external equipment when the test subject is ambulatory and unanesthetized, the information comprising the detected voltage difference or a signal derived from the detected voltage difference.
